(19) United States
(12) Patent Application Publication
(10) Pub. No.: US 2009/0012320 A1
(43) Pub. Date: Jan. 8, 2009

(54) Method for the Production of Olefins from Carbonyl Compounds

(75) Inventors: Benjamin List, Mulheim an der Ruhr (DE); Maria Hechavarria-Fonseca, Burstadt (DE); Arno Dohring, Mulheim an der Ruhr (DE); Andreas Job, Koln (DE)

Correspondence Address: NORRIS, MCLAUGHLIN & MARCUS, PA, 875 THIRD AVENUE, 18TH FLOOR, NEW YORK, NY, 10022, US

(73) Assignee: STUDIENGESELLSCHAFT KOHLE MBH, Mulheim an der Ruhr (DE)

(21) Appl. No.: 11/913,706

(22) PCT Filed: May 9, 2006
(86) PCT No.: PCT/DE06/00796

§ 371 Date: Nov. 6, 2007

(30) Foreign Application Priority Data
May 10, 2005 (DE) 10 2005 022 361.3

(51) Int. Cl. C07D 307/28; C07C 69/533; C07C 69/73; C07C 255/50

(52) U.S. Cl. 549/499; 560/205; 560/101; 560/104; 560/100; 560/174; 560/183; 560/75; 558/414; 560/51; 560/55; 560/128

(57) ABSTRACT

Carbonyl compounds of the formula (II), wherein R¹ and R² are as defined herein, react in the presence of an amine with carboxylic acid derivatives of the formula (II), wherein R³ and EWG are also as defined herein, to give α,β-unsaturated compounds of the formula (I) according to the following scheme:

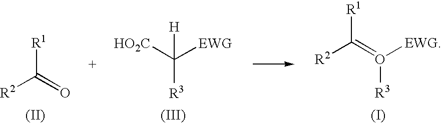

It is possible under mild reaction conditions to obtain unsaturated esters with high (E) stereoselectivity. The reaction typically proceeds at room temperature or lower without particular requirements such as inert gas, exclusion of moisture, heat, etc., being made. The only by-products obtained are CO₂ and water.

The present invention relates to a process for preparing olefins from carbonyl compounds.

The olefination of carbonyl compounds of the formula (1) with phosphorus compounds of the formula (2) is a known synthesis method for preparing unsaturated carbonyl compounds and similar compounds (equation 1, EWG=electron-withdrawing group).

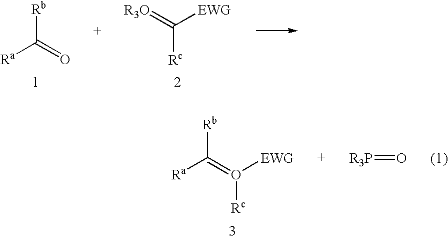

The most frequently used methods include the Wittig reaction (PR₃=PPh₃) and the Horner-Wadsworth-Emmons reaction (PR₃=P(OM) (OEt)₂). The two reactions are utilized to prepare relatively small amounts in research laboratories and also for commercial purposes with good yields and high selectivity. However, a disadvantage is that by-products are obtained in stoichiometric amounts in the reaction. As well as the desired reaction product, the Wittig reaction forms one equivalent of triphenylphosphine oxide (Ph₃PO), and the Horner-Wadsworth-Emmons reaction a phosphate salt (PO(OEt)₂OM). The two reaction by-products constitute a considerable problem in the case of production on the industrial scale, since these compounds have to be removed from the desired product and then disposed of or reprocessed. A further disadvantage for industrial scale production is that, in the Horner-Wadsworth-Emmons reaction, stoichiometric amounts of a base and in many cases also air- and moisture-sensitive compounds such as n-BuLi, LDA or NaH have to be used.

In an alternative process for synthesizing α,β-unsaturated esters and carboxylic acids, or in rarer cases also ketones, monoesters of malonic acid or similar compounds are reacted with carbonyl compounds (Galat-Doebner-Knoevenagel reaction) to obtain water and CO₂ as by-products (see equation 2).

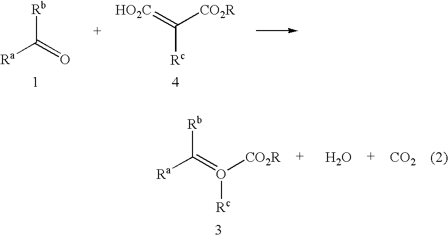

These reactions, which are referred to as modifications of the Knoevenagel reaction, are performed typically in pyridine as a solvent and in the presence of piperidine as a basic catalyst and at elevated temperature (>50° C.).

The stereoselectivity is typically lower than in the Wittig or Horner-Wadsworth-Emmons reaction, and both the α,β-unsaturated esters or acids desired here and the undesired α,β-unsaturated esters or acids, and also their mixtures, are isolated when enolizable carbonyl compounds are used. For example, the reaction of hexanal with malonic monoesters in different organic solvents and in the presence of a catalytic amount of piperidinium acetate under reflux affords the α,β-unsaturated ester as the main product. Apparently owing to the poorer E/Z and α,β vs. β,γ selectivity and owing to the reaction conditions (elevated temperature), the Galat-Doebner-Knoevenagel reaction is used to a lower degree than the Horner-Wadsworth-Emmons reaction.

It is an object of the present invention to provide a process for preparing α,β-unsaturated carbonyl compounds and related compounds, which does not have the disadvantages of the known reactions, specifically the formation of large amounts of by-products and strict requirements on the reaction conditions.

The present invention provides a process for preparing α,β-unsaturated compounds of the general formula I

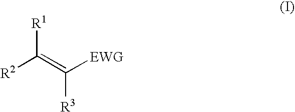

in which
R¹ and R² may be the same or different and are each hydrogen, substituted or unsubstituted alkyl or substituted or unsubstituted aryl,
R³ is hydrogen, substituted or unsubstituted alkyl or substituted or unsubstituted aryl, or a functional group, for instance OR⁴, NR⁵R⁶, SR⁷, where R⁴, R⁵, R⁶ and R⁷ may be customary substituents, especially alkyl and/or aryl groups, or halogen,
EWG may be an electron-withdrawing functional group, for example CO₂H, CO₂R⁸, CONR⁹R¹⁰, COSR¹¹, CN, NO₂, SO₂R¹²CHO, COR¹³, etc., where R⁸, R⁹, R¹⁰, R¹¹, R¹² and R¹³ may be customary substituents, especially alkyl and/or aryl groups,
in which a carbonyl compound of the formula II

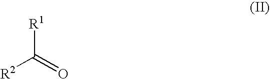

in which R¹ and R² are each as defined above,
in the presence of an amine, are reacted with a carboxylic acid of the formula III

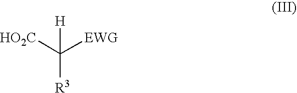

or with the same carboxylic acid generated in situ by adding an acid to its salt, in which R³ and EWG are each as defined above.

For example, it has been found that the reaction of aldehydes with carboxylic acids, such as malonic mono-esters, in the presence of an amine as a catalyst, can afford the corresponding unsaturated esters under mild reaction conditions with high (E) stereoselectivity. The process according to the invention is a catalytic reaction which proceeds typically at room temperature or lower without particular requirements such as inert gas, exclusion of moisture, heat, etc. have to be made. The only by-products obtained are CO₂ and water.

The term “alkyl” used means a linear, branched or cyclic hydrocarbon radical which has typically from 1 to 30, preferably from 1 to 24 carbon atoms, and especially from 1 to 6 carbon atoms, such as methyl, ethyl, n-propyl, isopropyl, n-butyl, isobutyl, t-butyl, octyl, decyl, etc., but also cycloalkyl groups such as cyclopentyl, cyclohexyl, etc. The hydrocarbon radicals have preferably from 1 to 18, especially from 1 to 12 carbon atoms.

The aryl groups used in the context of the present invention are aromatic ring systems having from 5 to 30 carbon atoms and optionally heteroatoms such as N, O, S, P, Si in the ring, and the rings may be single or multiple ring systems, for example fused ring systems rings bonded to one another via single bonds or multiple bonds. Examples of aromatic rings are phenyl, naphthyl, biphenyl, diphenyl ether, diphenylamine, benzophenone and the like. Substituted aryl groups have one or more substituents. Examples of heteroalkyl groups are alkoxyaryl, alkylsulfanyl-substituted alkyl, N-alkylated aminoalkyl and the like. Examples of heteroaryl substituents are pyrrolyl, pyrrolidinyl, pyridinyl, quinolinyl, indolyl, pyrimidinyl, imidazolyl, 1,2,4-triazolyl, tetrazolyl and the like. Examples of heteroatom-containing alicyclic groups include pyrrolidino, morpholino, piperazino, piperidino, etc.

The substituents that the aforementioned groups may have include, OH, F, Cl, Br, I, CN, NO₂, NO, SO₂, SO₃⁻, amino, —COOH, —COO(C₁-C₆-alkyl), mono- and di-(C₁-C₂₄-alkyl)-substituted amino, mono- and di-(C₅-C₂₀-aryl)-substituted amino, imino may be mentioned, which may in turn be substituted, for example C₁-C₆-alkyl, aryl and phenyl. Especially the cyclic radicals may also have C₁-C₆-alkyl groups as substituents.

The process according to the invention is performed in the presence of an amine as a catalyst. The amines used may be primary, secondary and tertiary amines, preference being given to cyclic amines such as DBU, DBN, DABCO, pyridine, piperidine, imidazole and derivatives thereof, and also aniline and derivatives thereof and mixtures of amines. It has been found that dimethylaminopyridines are particularly suitable, such as 4-dimethylaminopyridine (DMAP). The amine acts as a catalyst and is used in the process according to the invention preferably in an amount of from 0.1 to 15 mol %, especially from 5 to 10 mol %, based on the amount of the compound of the formula II or III.

The process according to the invention has the advantage that the reaction can be performed under mild reaction conditions. The reaction temperature may be from 0 to 30° C., preferably from 10 to 25° C. It is not necessary to perform the reaction under inert gas atmosphere or with exclusion of moisture.

In a preferred embodiment, the process is performed in an organic solvent. Useful solvents are those which do not adversely affect the reaction, such as pentane, hexane, heptane, octane, petroleum ether, toluene, xylenes, ethyl acetate, tetrahydrofuran, diethyl ether, methyl tert-butyl ether, 1,4-dioxane, methylene chloride, chloroform, carbon tetrachloride, dimethyl-formamide, sulfolane, 1,2-dichloroethane.

### EXAMPLES

### Reactions with the Monoester (General Method)

The reactions are performed in 5 ml glass vessels. 4-Dimethylaminopyridine (24.4 mg, 0.2 mmol) was dissolved in 5 ml of DMF, and aldehyde (2 mmol) and then the monoester (3 mmol) were added to the reaction. After a short time, the evolution of CO₂ was observed. The reaction was worked up after 5-60 h, the reaction mixture was extracted with diethyl ether and the organic phase was washed with NH₄Cl solution, H₂O, NaHCO₃ solution and finally with H₂O again. The organic phase was dried over Na₂SO₄, filtered and concentrated by rotary evaporation. In most cases, the crude product after this kind of workup had a purity of over 95%. All compounds were characterized fully by means of ¹H NMR, ¹³C NMR and HR-MS.

In the case of aromatic aldehydes or sterically hindered aldehydes, for example pivalaldehyde, the reaction time is shortened significantly in the case of addition of piperidine (17 mg, 0.2 mmol). For this purpose, the overall reaction mixture was cooled briefly (approx. 10° C.) and piperidine was added drop-wise, then stirring was continued at room temperature.

### Ethyl 2-Heptenoate

4-Dimethylaminopyridine (24.4 mg, 0.2 mmol) was dissolved in 5 ml of DMF, pentanal (172.3 mg, 2 mmol) and monoethyl malonate (396.4 mg, 3 mmol) were added to the reaction, and the mixture was stirred at 10° C. for 60 hours. After the aqueous workup, the ester was obtained as a colorless oil in 91% yield (284 mg, 1.82 mmol, E/Z=95:5).

### Ethyl 3-Cyclohexyl-2-Propenoate

4-Dimethylaminopyridine (24.4 mg, 0.2 mmol) was dissolved in 5 ml of DMF, cyclohexanecarboxaldehyde (224.3 mg, 2 mmol) and monoethyl malonate (396.4 mg, 3 mmol) were added to the reaction, and the mixture was stirred at room temperature for 48 hours. After the aqueous workup, the ester was obtained as a colorless oil in 92% yield (335.4 mg, 1.84 mmol, E/Z=98:2).

### Ethyl 4-Methyl-2-Pentenoate

4-Dimethylaminopyridine (24.4 mg, 0.2 mmol) was dissolved in 5 ml of DMF, isobutyraldehyde (144.2 mg, 2 mmol) and monoethyl malonate (396.4 mg, 3 mmol) were added to the reaction, and the mixture was stirred at room temperature for 16 hours. After the aqueous workup, the ester was obtained as a colorless oil in 96% yield (273.2 mg, 1.92 mmol, E/Z=99:1).

Ethyl 4,4-Dimethyl-2-Pentenoate

4-Dimethylaminopyridine (24.4 mg, 0.2 mmol) was dissolved in 5 ml of DMF, pivalaldehyde (172.1 mg, 2 mmol) and monoethyl malonate (396.4 mg, 3 mmol) were added to the reaction, and the mixture was stirred at room temperature for 60 hours. After the aqueous workup, the ester was obtained as a colorless oil in 92% yield (286.4 mg, 1.83 mmol, E/Z=99:1).

### Benzyl Cinnamate

4-Dimethylaminopyridine (24.4 mg, 0.2 mmol) was dissolved in 5 ml of DMF, benzaldehyde (210 mg, 2 mmol) and monobenzyl malonate (582 mg, 3 mmol) were added to the reaction, and the mixture was stirred at room temperature for five hours. After the aqueous workup, the benzyl cinnamate was obtained as a yellowish oil in 96% yield (452 mg, 1.9 mmol, E/Z=99:1).

### Ethyl P-Methoxyphenyl-2-Propenoate

4-Dimethylaminopyridine (24.4 mg, 0.2 mmol) were dissolved in 5 ml of DMF, and anisaldehyde (272.3 mg, 2 mmol) and monoethyl malonate (396.4 mg, 3 mmol) were added to the reaction. The mixture was cooled (10° C.) in order to slowly add the piperidine (17 mg, 0.2 mmol) dropwise. After the piperidine addition, stirring was continued at room temperature for 24 hours. After the aqueous workup, the ester was obtained as a yellow oil in quantitative yield (412.5 mg, 2 mmol, E/Z=99:1).

Reaction with the Potassium Salt of Monoethyl Malonate with Addition of Acids

### A) Addition of Hydrochloric Acid

4-Dimethylaminopyridine (24.4 mg, 0.2 mmol) was dissolved in 5 ml of DMF, and monoethyl malonate potassium salt (510.6 mg, 3 mmol) and, immediately thereafter, a solution of HCl in diethyl ether (1N, 3 ml) were added. The anisaldehyde (272.3 mg, 2 mmol) was then added. The mixture was cooled (10° C.) in order to slowly add the piperidine (17 mg, 0.2 mmol) dropwise. After the piperidine addition, stirring was continued at room temperature for 24 hours. After the aqueous workup, the ester was obtained as a yellow oil in 96% yield (409.3 mg, 1.98 mmol, E/Z=99:1).

### B) Addition of Acetic Acid

4-Dimethylaminopyridine (24.4 mg, 0.2 mmol) was dissolved in 5 ml of DMF, and monoethyl malonate potassium salt (510.6 mg, 3 mmol) and, immediately thereafter, a solution of acetic acid (180.2 mg, 3 mmol) were added. The anisaldehyde (272.3 mg, 2 mmol) was then added. The mixture was cooled (10° C.) in order to slowly add the piperidine (17 mg, 0.2 mmol) dropwise. After the piperidine addition, stirring was continued at room temperature for 24 hours. After the aqueous workup, the ester was obtained as a yellow oil in quantitative yield (412.5 mg, 2 mmol, E/Z=99:1).

(3)

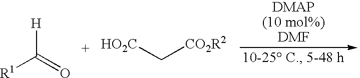

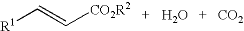

| Entry | R¹ | R² | Yield^{a} | E:Z^{b} |
|---|---|---|---|---|
| 1^{c} | 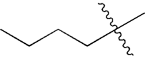 | Et | 91 | 95:5 |
| 2^{c} | 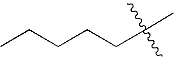 | Et | 95 | 96:4 |
| 3^{c} | 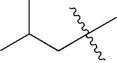 | Et | 90 | 95:5 |
| 4^{c} | 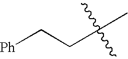 | Et | 91 | 95:5 |
| 5^{c} | 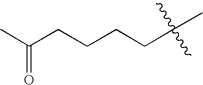 | Et | 91 | 98:2 |
| 6 | 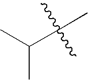 | Et | 96 | >99:1 |
| 7^{d} | 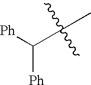 | Et | 91 | >99:1 |
| 8 | 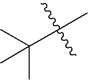 | Et | 92 | >99:1 |
| 9^{d} | | Et | 92 | >99:1 |
| 10^{d} | 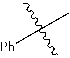 | t-Bu | 87 | 99:1 |
| 11^{d} | | Bn | 96 | >99:1 |
| 12^{d} | 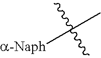 | Et | 92 | >99:1 |
| 13 | 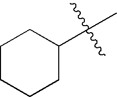 | Et | 92 | 98:2 |
| 14^{d} | 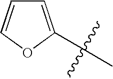 | Et | 90 | >99:1 |
| 15 | 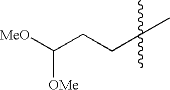 | Et | 92 | 94:6 |
| 16^{d} | 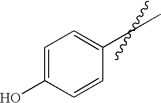 | Et | 90 | >99:1 |
| 17^{d} | 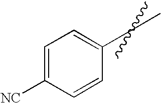 | Et | 94 | >99:1 |
| 18^{d,e} | 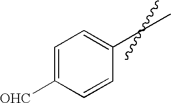 | Et | 93 | >99:1 |
| 19^{d}^{ₒ} | 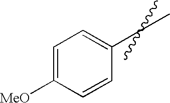 | Et | >99 | 99:1 |

^{a}Isolated yield.
^{b}Determined by GC.
^{c}Reaction at 10° C.
^{d}10 mol % of piperidine as a cocatalyst.
^{e}bis-Enoate with 3 equivalents of monoester.

1. A process for preparing α,β-unsaturated compounds of the general formula I: 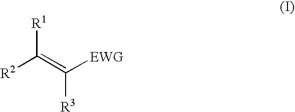
in which
R¹ and R² may be the same or different and are each hydrogen, substituted or unsubstituted alkyl or substituted or unsubstituted aryl,
R³ is hydrogen, substituted or unsubstituted alkyl or substituted or unsubstituted aryl, or a functional group,
EWG is an electron-withdrawing functional group,
comprising reacting a compound of the formula II: 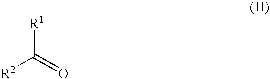
in which R¹ and R² are each as defined above,
in the presence of an amine, with a carboxylic acid derivative of the formula III: 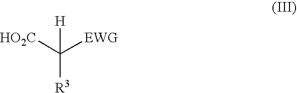
in which R³ and EWG are each as defined above.

2. The process as claimed in claim 1, wherein the amine is selected from the group consisting of primary, secondary and tertiary amines.

3. The process as claimed in claim 2, wherein the amine is 4-dimethylaminopyridine.

4. The process as claimed in claim 1, wherein said reacting is performed within a temperature range of from 0° C. to 30° C.

5. The process as claimed in claim 1, wherein the process is performed in an organic solvent.

6. The process as claimed in claim 1, wherein the carboxylic acid derivative of the formula III is obtained in situ from its salt by adding an acid.

7. The process as claimed in claim 1, wherein R³ is a functional group selected from the group consisting of OR⁴, NR⁵R⁶ and SR⁷, where R⁴, R⁵, R⁶ and R⁷ are independently selected from the group consisting of alkyl, halogen and aryl; and/or EWG is an electron-withdrawing functional group selected from the group consisting of CO₂H, CO₂R, CONR⁹R¹⁰, COSR¹¹, CN, NO₂, SO₂R¹², CHO and COR¹³ R⁸, R⁹, R¹⁰, R¹¹, R¹² and R¹³ are independently selected from the group consisting of alkyl and aryl.

8. The process as claimed in claim 2, wherein the amine is a cyclic amine.

9. The process as claimed in claim 8, wherein the cyclic amine is selected from the group consisting of pyridine, piperidine and derivatives thereof.
